(19) United States
(12) Patent Application Publication
(10) Pub. No.: US 2024/0197585 A1
(43) Pub. Date: Jun. 20, 2024

(54) ORAL CARE COMPOSITIONS

(71) Applicant: Colgate-Palmolive Company, New York, NY (US)

(72) Inventors: Shaoyi ZHANG, South Bound Brook, NJ (US); Suman CHOPRA, Monroe, NJ (US); Sergio LEITE, Monmouth Junction, NJ (US)

(73) Assignee: Colgate-Palmolive Company, New York, NY (US)

(21) Appl. No.: 18/544,959

(22) Filed: Dec. 19, 2023

Related U.S. Application Data
(60) Provisional application No. 63/476,023, filed on Dec. 19, 2022.

(51) Int. Cl. A61K 8/22; A61K 8/19; A61K 8/21; A61Q 11/00

(52) U.S. Cl. CPC A61K 8/22; A61K 8/19; A61K 8/21; A61Q 11/00; A61K 2800/48

(57) ABSTRACT

Aspects of the disclosure are directed to oral care compositions. In accordance with an aspect of the invention, provided is an oral care composition including from about 2 to about 10 wt. % of a peroxide source; about 5 wt. % or more of potassium nitrate; and about 45 wt. % or more of water, wherein all weight percentages are based on the total weight of the oral care composition.

### CROSS-REFERENCE TO RELATED APPLICATIONS

This application claims the benefit of priority from U.S. Provisional Application No. 63/476,023, filed Dec. 19, 2022, the contents of which are hereby incorporated herein by reference in their entirety.

### BACKGROUND

Oral care compositions, such as dentifrice compositions, can typically include one or more ingredients for maintaining the health of the oral cavity. Consumers have become aware of and desiring oral care compositions that also maintain the aesthetics of their teeth. It can be desirable to have dentifrice compositions with a relatively high level of peroxide of at least about 1 wt. % of the total weight of the oral care composition to achieve high whitening efficacy. However, it can be challenging to formulate dentifrice compositions with a relatively high level of peroxide because peroxide can be reactive with other dentifrice components, such as fluoride, metal ions, abrasives, etc.

Oral care compositions containing a relatively high level of peroxide have been particularly difficult to formulate with abrasives and non-insignificant amounts of water. One approach to the formulation challenges associated with incorporating sufficient peroxide in a dentifrice composition can be to separately package and/or apply a peroxide separate from the dentifrice composition. Unfortunately, consumer compliance can be lower when a peroxide must be applied separately from the dentifrice composition.

As such, there is a need for an improved oral care composition having enhance peroxide stability.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions. In accordance with an aspect of the invention, provided is an oral care composition including from about 2 to about 10 wt. % of a peroxide source; about 5 wt. % or more of potassium nitrate; and about 45 wt. % or more of water, wherein all weight percentages are based on the total weight of the oral care composition.

According to another aspect of the invention, provided is an oral care composition including from about 2 wt. % to about 10 wt. % of a peroxide source; about 5 wt. % or more of potassium nitrate; from about 1 to about 35 wt. % of an abrasive comprising calcium pyrophosphate; from about 0.5 to about 10 wt. % of an anionic surfactant; and from about 50 to about 75 wt. % or more of water, wherein all weight percentages are based on the total weight of the oral care composition.

In accordance to a further aspect of the invention, provided is an oral care composition from about 2 wt. % to about 10 wt. % of a peroxide source; from about 1 to about 35 wt. % of an abrasive comprising calcium pyrophosphate; optionally, from about 0.5 to about 10 wt. % of an anionic surfactant; a thickening agent selected from a carbopol, carboxymethylcellulose, a silica thickener, polyacrylate crosspolymer-6, and a combination of two or more thereof; and from about 45 to about 80 wt. % or more of water, wherein all weight percentages are based on the total weight of the oral care composition.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “∘” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a fluoride ion source and a stannous ion source. If a particular claim to an oral care composition includes both a polyol and a humectant, glycerin will serve only as either a polyol or a humectant—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions having enhanced stability. In accordance with an aspect of the invention, provided is an oral care composition including from about 2 to about 10 wt. % of a peroxide source; about 5 wt. % or more of potassium nitrate; and about 45 wt. % or more of water, wherein all weight percentages are based on the total weight of the oral care composition.

According to another aspect of the invention, provided is an oral care composition including from about 2 wt. % to about 10 wt. % of a peroxide source; about 5 wt. % or more of potassium nitrate; from about 1 to about 35 wt. % of an abrasive comprising calcium pyrophosphate; from about 0.5 to about 10 wt. % of an anionic surfactant; and from about 50 to about 75 wt. % or more of water, wherein all weight percentages are based on the total weight of the oral care composition.

In accordance to a further aspect of the invention, provided is an oral care composition from about 2 wt. % to about 10 wt. % of a peroxide source; from about 1 to about 35 wt. % of an abrasive comprising calcium pyrophosphate; optionally, from about 0.5 to about 10 wt. % of an anionic surfactant; a thickening agent selected from a carbopol, carboxymethylcellulose, a silica thickener, polyacrylate crosspolymer-6, and a combination of two or more thereof; and from about 45 to about 80 wt. % or more of water, wherein all weight percentages are based on the total weight of the oral care composition.

The oral care compositions may be in the form of a dentifrice, a toothpaste, a gel, and/or the like. Suitable components, such as those listed below, may be included or excluded from the formulations for the oral care compositions disclosed herein depending on the specific combination of other ingredients and/or the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

Typically, the oral care compositions include one or more whitening agent(s) comprising a peroxide, preferably in an effective amount. The amount of whitening agent(s) present in the oral care composition may be from about 1 to about 30 wt. %, based on the total weight of the oral care composition. For example, the whitening agent(s) may be present in the oral care composition in an amount from about 1 to about 30 wt. %, about 1 to about 26 wt. %, about 1 to about 22 wt. %, about 1 to about 19 wt. %, about 1 to about 16 wt. %, about 1 to about 13 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 30 wt. %, about 2 to about 26 wt. %, about 2 to about 22 wt. %, about 2 to about 19 wt. %, about 2 to about 16 wt. %, about 2 to about 13 wt. %, about 2 to about 10 wt. %, about 2 to about 8 wt. %, about 2 to about 6 wt. %, about 2 to about 5 wt. %, about 2 to about 4 wt. %; from about 3 to about 30 wt. %, about 3 to about 26 wt. %, about 3 to about 22 wt. %, about 3 to about 19 wt. %, about 3 to about 16 wt. %, about 3 to about 13 wt. %, about 3 to about 10 wt. %, about 3 to about 8 wt. %, about 3 to about 6 wt. %; from about 5 to about 30 wt. %, about 5 to about 26 wt. %, about 5 to about 22 wt. %, about 5 to about 19 wt. %, about 5 to about 16 wt. %, about 5 to about 13 wt. %, about 5 to about 10 wt. %, about 5 to about 8 wt. %; from about 8 to about 30 wt. %, about 8 to about 26 wt. %, about 8 to about 22 wt. %, about 8 to about 19 wt. %, about 8 to about 16 wt. %, about 8 to about 13 wt. %; from about 11 to about 30 wt. %, about 11 to about 26 wt. %, about 11 to about 22 wt. %, about 11 to about 19 wt. %, about 11 to about 16 wt. %; from about 14 to about 30 wt. %, about 14 to about 26 wt. %, about 14 to about 22 wt. %, about 14 to about 19 wt. %; from about 18 to about 30 wt. %, about 18 to about 26 wt. %, about 18 to about 22 wt. %, or any range or subrange thereof, based on the total weight of the oral are composition.

The whitening agent(s) typically comprise a peroxide, such as hydrogen peroxide, peroxides of alkali and alkaline earth metals, organic peroxy compounds, peroxy acids, salts thereof, or combinations of two or more thereof. Peroxides of alkali and alkaline earth metals include lithium peroxide, potassium peroxide, sodium peroxide, magnesium peroxide, calcium peroxide, barium peroxide, and a combination of two or more thereof. Examples of organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate, or a combination of two or more thereof. Peroxy acids and their salts include organic peroxy acids, such as alkyl peroxy acids, monoperoxyphthalate, and a combination thereof as well as inorganic peroxy acid salts, such as persulfate, dipersulfate, percarbonate, perphosphate, perborate and persilicate salts of alkali and/or alkaline earth metals, such as lithium, potassium, sodium, magnesium, calcium and barium, and mixtures thereof. In various embodiments, the peroxide compound comprises hydrogen peroxide, urea peroxide, sodium percarbonate, or a combination of two or more thereof.

The one or more peroxide(s) may be a non-complexed peroxide. The non-complexed peroxide may be chosen from one or more of the peroxides described above. In some preferred embodiments, the non-complexed peroxide comprises hydrogen peroxide.

The total amount of whitening agent may comprise from about 0.01 to about 10 wt. % of a non-complexed peroxide source, based on the total weight of the oral care composition. For example, the whitening agent may comprise a non-complexed peroxide source in an amount from about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %; from about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; from about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %, or any range or subrange thereof, based on the total weight of the oral care composition.

In some embodiments, however, the whitening agent may comprise a peroxide complex. The peroxide complex may comprise a peroxide component and a porous cross-linked polymer. As referred to herein, a “peroxide component” is any oxidizing compound comprising a bivalent oxygen-oxygen group. Peroxide components include peroxides and hydroperoxides disclosed above, such as hydrogen peroxide, peroxides of alkali and alkaline earth metals, organic peroxy compounds, peroxy acids, salts thereof, and mixtures thereof. In various embodiments, the peroxide component comprises hydrogen peroxide, urea peroxide, sodium percarbonate and mixtures thereof. In one embodiment, the peroxide component comprises hydrogen peroxide. In one embodiment, the peroxide component consists essentially of hydrogen peroxide.

The whitening agent may comprise a peroxide complex, such as PVP complexed with hydrogen peroxide, cross-linked PVP complexed with hydrogen peroxide, silica complexed with hydrogen peroxide, tetrasodium pyrophosphate complexed with hydrogen peroxide, or a combination of two or more thereof. The peroxide complex may, also or alternatively, be selected from N-vinyl heterocyclic polymers. Preferably, the N-vinyl heterocyclic polymer is crosslinked and adsorbs, absorbs, complexes, or otherwise retains the peroxide component. The chemical and physical characteristics of the particulate hinder the release of the peroxide compound from the polymer particulates, and in some embodiments provides controlled release of the peroxide compound. The peroxide complex preferably comprises a peroxide component at a level of from about 0.1 to about 25 wt. %, from about 1 to about 25 wt. %, from about 5 to about 18 wt. %, based on the weight of the peroxide complex. In some preferred embodiments, the oral care composition comprises a complex of peroxide adsorbed to cross-linked polyvinylpyrrollidone.

In some embodiments, a non-peroxide whitening agent may be included in the oral care composition. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments or dyes.

In some preferred embodiments, the oral care composition includes about 2 to about 10 wt. % or more of potassium nitrate, based on the total weight of the oral care composition. For example, the oral care composition may include about 2 to about 8 wt. %, about 2 to about 7 wt. %, about 2 to about 6 wt. %, about 2 to about 5.5 wt. %, about 2 to about 5 wt. %, about 2 to about 4.5 wt. %; from about 3 to about 10 wt. %, about 3 to about 8 wt. %, about 3 to about 7 wt. %, about 3 to about 6 wt. %, about 3 to about 5.5 wt. %, about 3 to about 5 wt. %, about 3 to about 4.5 wt. %; from about 4 to about 10 wt. %, about 4 to about 8 wt. %, about 4 to about 7 wt. %, about 4 to about 6 wt. %, about 4 to about 5.5 wt. %, about 4 to about 5 wt. %; from about 4.5 to about 10 wt. %, about 4.5 to about 8 wt. %, about 4.5 to about 7 wt. %, about 4.5 to about 5.5 wt. %; from about 5 to about 10 wt. %, about 5 to about 8 wt. %, about 5 to about 7 wt. %, about 5 to about 6 wt. %; from about 6 to about 10 wt. %, about 6 to about 8 wt. %, about 6 to about 7 wt. %; from about 7 to about 10 wt. %, about 7 to about 8 wt. %, or any range or subrange thereof, based on the total weight of the oral care composition. In certain embodiments, the oral care composition includes about 5 wt. % of potassium nitrate, based on the total weight of the oral care composition.

The oral care compositions may include one or more desensitizing agent other than potassium nitrate. The amount of desensitizing agent(s) other than potassium nitrate may be from about 0.5 to about 9 wt. %, based on the total weight of the oral care composition. For example, the amount of desensitizing agent(s) other than potassium nitrate in the oral care composition may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 6 wt. %, about 2 to about 5 wt. %, about 2 to about 4 wt. %, about 2 to about 3 wt. %; from about 3 to about 9 wt. %, about 3 to about 8 wt. %, about 3 to about 6 wt. %, about 3 to about 5 wt. %, about 3 to about 4 wt. %; from about 4 to about 9 wt. %, about 4 to about 8 wt. %, about 4 to about 6 wt. %, about 4 to about 5 wt. %; from about 5 to about 9 wt. %, about 5 to about 8 wt. %, about 5 to about 6 wt. %; from about 6 to about 9 wt. %, about 6 to about 8 wt. %; from about 7 to about 9 wt. %, about 7 to about 8 wt. %, about 8 to about 10 wt. %, or any range or subrange therefrom, based on the total weight of the oral care composition.

The desensitizing agent(s) other than potassium nitrate may be, e.g., benzocaine; potassium fluoride; strontium chloride; potassium chloride; potassium citrate; iron oxalate; sodium nitrate; lithium nitrate; magnesium nitrate; calcium nitrate; calcium hydroxide; dibasic calcium phosphate; strontium acetate; sodium monofluorophosphate; bisabolol; a local or systemic analgesic agent such as NSAIDS, aspirin, acetaminophen and/or codeine; or combinations of two or more thereof. For example, the desensitizing agent(s) may be chosen from potassium citrate, potassium chloride, potassium bicarbonate, potassium oxalate, and a combination of two or more thereof.

The oral care compositions may include one or more surfactant(s) in various amounts. The oral care composition may comprise one or more anionic surfactant, one or more cationic surfactant, one or more nonionic surfactant, or a combination of two or more thereof. For example, the oral care composition may include one or more anionic surfactant(s) in an amount of about 0.5 to about 10 wt. %, based on the total weight of the oral care composition. In some instances, the anionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anionic surfactant(s) may be selected from sulfate based anionic surfactants and non-sulfate based anionic surfactants. For example, the oral care composition may include an anionic surfactant chosen from ammonium lauryl sulfate, ammonium lauryl ether sulfate, sodium lauryl sulfate, sodium lauryl ether sulfate sodium dodecyl sulfate, sodium coco-sulfate, ammonium coco-sulfate, and a combination of two or more thereof. In some preferred embodiments, the oral care composition comprises sodium lauryl sulfate, sodium lauryl ether sulfate, or a combination thereof. In further embodiments, one or more anionic surfactant is selected from sodium lauryl sulfate, sodium lauryl ether sulfate, sodium lauryl sarcosinate, sodium cocoyl methyl taurate, sodium monoglyceride sulfate, sodium cetearyl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

The one or more anionic surfactant(s) may be selected from non-sulfate based anionic surfactants, such as sulfonated monoglycerides of fatty acids, acyl isethionates, acyl sarcosinates, acyl taurate, and a combination of two or more thereof. Non-limiting examples of acyl isethionates include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Examples of acyl taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the oral care composition comprises sodium lauroyl methyl taurate (or sodium methyl lauroyl taurate), sodium methyl cocoyl taurate (SMCT), or a combination thereof.

The anionic surfactant(s) may have at least one acyl group, preferably, comprising a carbon chain of 8 to 21 carbons. In some instance, the alkyl group(s) of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

Additionally or alternatively, the oral care composition may comprise one or more nonionic surfactant(s). The oral care composition may include nonionic surfactant(s) in an amount of about 0.5 to about 10 wt. %, based on the total weight of the oral care composition. In some instances, the nonionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl n₄₀₋₆₀ hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethylene glycol octylphenol ethers, sorbitan alkyl esters, polyoxyethylene glycol sorbitan alkyl esters, or a combination of two or more thereof.

In some embodiments, the oral care composition may comprise one or more cationic surfactant(s). The oral care composition may include cationic surfactant(s) in an amount of about 0.5 to about 10 wt. %, based on the total weight of the oral care composition. In some instances, the cationic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

Examples of cationic surfactants that may be present in the oral care composition include cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be present in the form of a salt.

In some embodiments, the oral care composition may include an emulsifier, e.g., in an amount from about 0.5 to about 10 wt. %, based on the total weight of the oral care composition. For example, the total amount of emulsifier(s) in the oral care composition may be from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The emulsifier may be chosen from natural emulsifiers, such as acacia, gelatin, lecithin and cholesterol; finely dispersed solids, such as colloidal clays, bentonite, veegum (magnesium aluminum silicate; and synthetic emulsifiers, such as salts of fatty acids, sulfates such as sorbitan trioleate, sorbitan tristearate, sucrose distearate, propylene glycol monostearate, glycerol monostearate, propylene glycol monolaurate, sorbitan monostearate, sorbitan monolaurate, poly oxy ethylene-4-lauryl ether, sodium lauryl sulfate, sulfonates such as dioctyl sodium sulfosuccinate, glyceryl esters, polyoxyethylene glycol esters and ethers, diethylene glycol monostearate, PEG 200 distearate, and sorbitan fatty acid esters, such as sorbitan monopalmitate, and their polyoxyethylene derivatives, polyoxyethylene glycol esters such as the monostearate, Polysorbate 80 (ethoxylated sorbitan monooleate) (supplied by Spectrum, etc.); and combinations of two or more thereof.

The emulsifier can comprise a polysorbate, an alkyl sulfate, Lipowax® D, or combinations thereof. Suitable polysorbate compounds include, polysorbate 20, 40, 60, 80, or combinations thereof, such as Tween® 20, 40, 60, 80, or combinations thereof.

The emulsifier can be a surfactant that is non-reactive with a whitening agent. For example, surfactants that are non-reactive with a bleaching agent may be substantially free of hydroxy groups, nitrogen groups and linkages, double or triple covalent bonds between adjacent carbon atoms, metals such as Zn, etc., or combinations thereof.

The oral care compositions may include one or more fatty alcohol(s). The amount of fatty alcohol present in the oral care composition may be from about 0.05 to about 7 wt. %, based on the total weight of the oral care composition. For example, the total amount of fatty alcohol(s) in the oral care composition may be from about 0.05 to about 7 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.8 wt. %, about 0.05 to about 0.5 wt. %; from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.5 wt. %; from about 0.3 to about 7 wt. %, about 0.3 to about 5 wt. %, about 0.3 to about 4 wt. %, about 0.3 to about 3 wt. %, about 0.3 to about 2 wt. %, about 0.3 to about 1 wt. %, about 0.3 to about 0.8 wt. %, about 0.3 to about 0.5 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.8 wt. %; from about 0.8 to about 7 wt. %, about 0.8 to about 5 wt. %, about 0.8 to about 4 wt. %, about 0.8 to about 3 wt. %, about 0.8 to about 2 wt. %, about 0.8 to about 1 wt. %; from about 1 to about 7 wt. %, about 1 to about 5 wt. %, about 1 to about 4 wt. %, about 1 to about 3 wt. %, about 1 to about 2 wt. %; from about 1.5 to about 7 wt. %, about 1.5 to about 5 wt. %, about 1.5 to about 4 wt. %, about 1.5 to about 3 wt. %; from about 2.5 to about 7 wt. %, about 2.5 to about 5 wt. %, about 2.5 to about 4 wt. %; from about 4 to about 7 wt. %, about 4 to about 5 wt. %; from about 5 to about 7 wt. %, or any range or subrange thereof, based on the total weight of the oral care composition.

The fatty alcohol(s) may be selected from decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, isotridecyl alcohol, myricyl alcohol, and a mixture of two or more thereof. In some cases, the surfactant system comprises cetyl alcohol, stearyl alcohol, cetearyl alcohol, lauryl alcohol, or a combination thereof.

The oral care compositions typically include an abrasive. The amount of abrasive(s) present in the oral are composition may be from about 1 to about 35 wt. %, based on the total weight of the oral care composition. In some instances, the total amount of abrasive(s) present in the oral care composition is from about 1 to about 35 wt. %, about 1 to about 35 wt. %, about 1 to about 31 wt. %, about 1 to about 27 wt. %, about 1 to about 23 wt. %, about 1 to about 19 wt. %, about 1 to about 17 wt. %, about 1 to about 15 wt. %, about 1 to about 13 wt. %; from about 4 to about 35 wt. %, about 4 to about 35 wt. %, about 4 to about 31 wt. %, about 4 to about 27 wt. %, about 4 to about 23 wt. %, about 4 to about 19 wt. %, about 4 to about 17 wt. %, about 4 to about 15 wt. %, about 4 to about 13 wt. %; from about 7 to about 35 wt. %, about 7 to about 35 wt. %, about 7 to about 31 wt. %, about 7 to about 27 wt. %, about 7 to about 23 wt. %, about 7 to about 19 wt. %, about 7 to about 17 wt. %, about 7 to about 15 wt. %, about 7 to about 13 wt. %; from about 10 to about 35 wt. %, about 10 to about 35 wt. %, about 10 to about 31 wt. %, about 10 to about 27 wt. %, about 10 to about 23 wt. %, about 10 to about 19 wt. %, about 10 to about 17 wt. %, about 10 to about 15 wt. %; from about 13 to about 35 wt. %, about 13 to about 35 wt. %, about 13 to about 31 wt. %, about 13 to about 27 wt. %, about 13 to about 23 wt. %, about 13 to about 19 wt. %, about 13 to about 17 wt. %, about 13 to about 15 wt. %; from about 15 to about 35 wt. %, about 15 to about 35 wt. %, about 15 to about 31 wt. %, about 15 to about 27 wt. %, about 15 to about 23 wt. %, about 15 to about 19 wt. %; from about 17 to about 35 wt. %, about 17 to about 35 wt. %, about 17 to about 31 wt. %, about 17 to about 27 wt. %, about 17 to about 23 wt. %; from about 19 to about 35 wt. %, about 19 to about 35 wt. %, about 19 to about 31 wt. %, about 19 to about 27 wt. %, about 19 to about 23 wt. %, or any range or subrange thereof, based on the total weight of the oral care composition.

The abrasive(s) may include one or more abrasives based on insoluble phosphate salts, such as those chosen from calcium pyrophosphate, dicalcium orthophosphate dihydrate, tricalcium phosphate, and calcium polymetaphosphate, and a combination of two or more thereof. In some embodiments, the abrasive is comprised primarily of calcium pyrophosphate and, in at least one embodiment, calcium pyrophosphate is the sole abrasive in the oral care composition. For example, the calcium pyrophosphate may comprise about 50 wt. % or more, about 60 wt. % or more, about 70 wt. % or more, about 80 wt. % or more, about 95 wt. % or more, about 98 wt. % or more, or about 99 wt. % or more of the total amount of the abrasive in the oral care composition.

The oral care compositions may include an anti-calculus agent. The amount of anti-calculus agent(s) present in the oral are composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The oral care compositions may include anti-calculus agents chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. For instance, the anti-calculus agent may comprise alkali-metal pyrophosphate, a hypophosphite-containing polymer, an organic phosphocitrate, a phosphocitrate, a polyphosphate, or a combination of two or more thereof. Examples of additional anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, such as tetrasodium pyrophosphate. In at least one embodiment, the anti-calculus agent(s) is selected from sodium acid pyrophosphate, tetrasodium pyrophosphate, and a combination of two or more thereof.

The oral care compositions may include one or more thickening agent(s) in an amount from about 0.5 to about 20 wt. %, based on the total weight of the oral care composition. In some embodiments, to total amount of thickening agent(s) in the oral care composition is from about 0.5 to about 17 wt. %, about 0.5 to about 15 wt. %, about 0.5 to about 13 wt. %, about 0.5 to about 11 wt. %, about 0.5 to about 9 wt. %, about 0.5 to about 7 wt. %, about 0.5 to about 5 wt. %; from about 1 to about 20 wt. %, about 1 to about 17 wt. %, about 1 to about 15 wt. %, about 1 to about 13 wt. %, about 1 to about 11 wt. %, about 1 to about 9 wt. %, about 1 to about 7 wt. %, about 1 to about 5 wt. %; from about 3 to about 20 wt. %, about 3 to about 17 wt. %, about 3 to about 15 wt. %, about 3 to about 13 wt. %, about 3 to about 11 wt. %, about 3 to about 9 wt. %, about 3 to about 7 wt. %; from about 5 to about 20 wt. %, about 5 to about 17 wt. %, about 5 to about 15 wt. %, about 5 to about 13 wt. %, about 5 to about 11 wt. %, about 5 to about 9 wt. %; from about 7 to about 20 wt. %, about 7 to about 17 wt. %, about 7 to about 15 wt. %, about 7 to about 13 wt. %; from about 10 to about 20 wt. %, about 10 to about 17 wt. %, about 10 to about 15 wt. %, about 10 to about 13 wt. %; from about 13 to about 20 wt. %, about 13 to about 17 wt. %; from about 16 to about 20 wt. %, or including ranges and subranges thereof, based on the total weight of the oral care composition.

The one or more thickening agent(s) may be selected from a silica thickener, a polymer, a fatty acid, a fatty amphiphile, and a combination of two or more thereof. Silica thickeners typically form a polymeric structure or gel in an aqueous media. The silica thickeners may be chosen from precipitated silica, fumed silica, hydrated silica, colloidal silica, a metal silicate, and combinations thereof. In some instances, the silica thickener(s) comprises fumed silica, colloidal silica, or a combination thereof. The silica thickener may include a metal silicate chosen from sodium silicate, potassium silicate, and a combination of two or more thereof. Additional examples of silica thickeners are disclosed in U.S. Pat. No. 6,342,205, which is incorporated herein by reference in its entirety for all purposes.

Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000 g/mol, preferably in the range 5,000 to 50,000 g/mol. The polyvinyl pyrrolidone may, in some embodiments, have an average molecular weight of ranging from about 8,000 to 12,000 g/mol (e.g., about 10,000 g/mol), from about 27,000 g/mol to about 33,000 g/mol (e.g., about 30,000 g/mol) and/or from about 37,000 g/mol to about 43,000 g/mol (e.g., about 40,000 g/mol). The polyvinyl pyrrolidone may be in the form of a hydrogen peroxide-polyvinyl pyrrolidone polymer complex. Examples of polyvinyl pyrrolidone complexes include those disclosed in U.S. Pat. No. 5,122,370, the contents of which are incorporated herein by reference for all purposes. In some embodiments, the polymer comprises crosslinked polyvinyl pyrrolidone (crosslinked PVP). In at least one embodiment, the polymer consists essentially of or consists of polyvinyl pyrrolidone.

The thickening agent(s) may be chosen from polysaccharides, such as xanthan gum, cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, and a combination of two more thereof.

Additionally or alternatively, the thickening agent(s) may be an acrylate. Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol. In some preferred embodiments, the one or more thickening agent(s) comprises polyacrylate crosspolymer-6.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylolpropane methacrylate, ethoxyethyl methacrylate, trifluoroethyl methacrylate, or a combination of two or more thereof.

In some embodiments, the one or more thickening agent(s) comprise acrylamides including, but are not limited to, acrylamide, methacrylamide and di(C1-C30) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, 2-acrylamido-2-methylpropanesulfonic acid, or a combination of two or more thereof.

The one or more thickening agent(s) may be selected from crosslinked polyacrylic acid polymer. In at least one embodiment, the oral care composition includes a carbopol.

In certain embodiments, the one or more thickening agent(s) may be chosen from an amphiphilic polymer. The amphiphilic polymer may be included as a peroxide compatible thickening agent. The amphiphilic polymer may comprise a polymer that has a hydrophobic portion and a hydrophilic portion. For example, the polymer may comprise a polymer macromolecule that comprises a hydrophilic backbone and hydrophobic subunits. This can allow the amphiphilic polymer to remain soluble in water while preventing strong interactions with the peroxide.

The amphiphilic polymer can include polymers that are at least partially soluble and/or fully soluble in water. Examples of amphiphilic polymers include 2-acrylamido-2-methylpropane sulfonic acid (AMPS) polymer, copolymer, cross-polymer, or combination thereof. In one example, the oral care composition can contain polyacrylate crosspolymer-6 (commercially available as SepiMAX™ ZEN from SEPPIC S.A., a subsidiary of the Air Liquide group, Puteaux Cedex, France).

The oral care compositions may include a fluoride ion source. The fluoride ion source may be present in an effective amount. In some cases, the fluoride ion source is present in the oral care composition in an amount of about 0.1 to about 7 wt. %, based on the total weight of the oral care composition. For example, the fluoride ion source may be present in an amount of about 0.1 to about 7 wt. %, about 0.1 to about 5 wt. %, about 0.1 to about 3 wt. %, about 0.1 to about 2 wt. %, about 0.1 to about 1 wt. %; from about 0.4 to about 7 wt. %, about 0.4 to about 5 wt. %, about 0.4 to about 3 wt. %, about 0.4 to about 2 wt. %, about 0.4 to about 1 wt. %; from about 0.8 to about 7 wt. %, about 0.8 to about 5 wt. %, about 0.8 to about 3 wt. %, about 0.8 to about 2 wt. %, about 0.8 to about 1.5 wt. %; from about 1.4 to about 7 wt. %, about 1.4 to about 5 wt. %, about 1.4 to about 3 wt. %; from about 2 to about 7 wt. %, about 2 to about 5 wt. %, about 2 to about 3 wt. %; from about 3 to about 7 wt. %, about 3 to about 5 wt. %, about 3 to about 4 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the oral care composition.

The oral care compositions may include one or more polyol(s). The one or more polyol(s) may be present in the oral care composition in an amount from about 1 to about 50 wt. %, based on the total weight of the oral care composition. For example, the oral care composition may include polyol(s) in an amount from about 1 to about 40 wt. %, about 1 to about 30 wt. %, about 1 to about 20 wt. %, about 1 to about 17 wt. %, about 1 to about 14 wt. %, about 1 to about 11 wt. %, about 1 to about 9 wt. %, about 1 to about 7 wt. %, about 1 to about 5 wt. %, about 1 to about 3 wt. %; from about 3 to about 50 wt. %, about 3 to about 40 wt. %, about 3 to about 30 wt. %, about 3 to about 20 wt. %, about 3 to about 17 wt. %, about 3 to about 14 wt. %, about 3 to about 11 wt. %, about 3 to about 9 wt. %, about 3 to about 7 wt. %; from about 6 to about 50 wt. %, about 6 to about 40 wt. %, about 6 to about 30 wt. %, about 6 to about 20 wt. %, about 6 to about 17 wt. %, about 6 to about 14 wt. %, about 6 to about 11 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %, about 10 to about 20 wt. %, about 10 to about 17 wt. %, about 10 to about 14 wt. %; from about 15 to about 50 wt. %, about 15 to about 45 wt. %, about 15 to about 40 wt. %, about 15 to about 35 wt. %, about 15 to about 30 wt. %, about 15 to about 25 wt. %, about 15 to about 20 wt. %; from about 20 to about 50 wt. %, about 20 to about 45 wt. %, about 20 to about 40 wt. %, about 20 to about 35 wt. %, about 20 to about 30 wt. %, about 20 to about 25 wt. %; from about 30 to about 50 wt. %, about 30 to about 45 wt. %, about 30 to about 40 wt. %; from about 40 to about 50 wt. %, about 40 to about 45 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The polyol may be a humectant chosen from glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer and a combination of two or more thereof.

The polyol(s) may be selected from glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof.

In some cases, the oral care composition includes one or more polyol selected from the group consisting of C2-C32 polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be incorporated in the oral care composition include alkanediols, such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers, such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600 (CAS-25322-68-3). In some embodiments, the oral care compositions comprise one or more polyethylene glycols, e.g., polyethylene glycols in a molecular weight range from 200 to 800. For example, the oral care composition may comprise polyethylene glycol 200, polyethylene glycol 300, polyethylene glycol 400, polyethylene glycol 600, polyethylene glycol 800, or a combination of two or more thereof.

In some embodiments, the oral care composition may include an amino acid, e.g., in an amount from about 0.01 to about 20 wt. %, based on the total weight of the oral care composition. For instance, the total amount of amino acid(s) in the oral care composition may be from about 0.1 to about 10 wt. %, from about 0.5 to about 6 wt. %, or from about 1 to about 10 wt. % of amino acid, by weight of the oral care composition.

The amino acid can comprise one or more amino acids, peptide, and/or polypeptide, as described herein. The amino acids may include one or more basic amino acids, one or more acidic amino acids, one or more neutral amino acids, or combinations thereof.

Amino acids are typically organic compounds that contain an amine functional group, a carboxyl functional group, and a side chain specific to each amino acid. Examples of amino acids include amino acids with a positive or negative side chain, amino acids with an acidic or basic side chain, amino acids with polar uncharged side chains, amino acids with hydrophobic side chains, and/or combinations thereof. The amino acids may be chosen from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion sources are disclosed in U.S. Pat. Nos. 3,535,421, 4,885,155, and 3,678,154, the disclosures of which are hereby incorporated by reference in their entirety for all purposes.

In some embodiments, the oral care compositions may include one or more sweeteners. The oral care composition may include caloric sweeteners and/or non-caloric sweeteners. Examples of non-caloric sweeteners include saccharin, e.g., sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 7 wt. %, based on the total weight of the oral care composition. For example, the oral care composition may have a total amount of sweetener(s) from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %; from about 4 to about 7 wt. %, about 4 to about 5 wt. %; from about 5 to about 7 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges thereof, based on the total weight of the oral care composition.

The oral care composition may comprise from about 10 to about 90 wt. % of water, based on the total weight of the oral care composition. In some embodiment, the amount of water in the oral care composition is from about 10 to about 90 wt. %, about 10 to about 80 wt. %, about 10 to about 75 wt. %, about 10 to about 70 wt. %, about 10 to about 67 wt. %, about 10 to about 64 wt. %, about 10 to about 61 wt. %, about 10 to about 58 wt. %, about 10 to about 55 wt. %, about 10 to about 53 wt. %, about 10 to about 51 wt. %, about 10 to about 48 wt. %, about 10 to about 45 wt. %, about 10 to about 40 wt. %; from about 20 to about 90 wt. %, about 20 to about 80 wt. %, about 20 to about 75 wt. %, about 20 to about 70 wt. %, about 20 to about 67 wt. %, about 20 to about 64 wt. %, about 20 to about 61 wt. %, about 20 to about 58 wt. %, about 20 to about 55 wt. %, about 20 to about 53 wt. %, about 20 to about 51 wt. %, about 20 to about 48 wt. %, about 20 to about 45 wt. %, about 20 to about 40 wt. %; from about 30 to about 90 wt. %, about 30 to about 80 wt. %, about 30 to about 75 wt. %, about 30 to about 70 wt. %, about 30 to about 67 wt. %, about 30 to about 64 wt. %, about 30 to about 61 wt. %, about 30 to about 58 wt. %, about 30 to about 55 wt. %, about 30 to about 53 wt. %, about 30 to about 51 wt. %, about 30 to about 48 wt. %, about 30 to about 45 wt. %; from about 40 to about 90 wt. %, about 40 to about 80 wt. %, about 40 to about 75 wt. %, about 40 to about 70 wt. %, about 40 to about 67 wt. %, about 40 to about 64 wt. %, about 40 to about 61 wt. %, about 40 to about 58 wt. %, about 40 to about 55 wt. %; from about 45 to about 90 wt. %, about 45 to about 80 wt. %, about 45 to about 75 wt. %, about 45 to about 70 wt. %, about 45 to about 67 wt. %, about 45 to about 64 wt. %, about 45 to about 61 wt. %; from about 50 to about 90 wt. %, about 50 to about 80 wt. %, about 50 to about 75 wt. %, about 50 to about 70 wt. %, about 50 to about 67 wt. %, about 50 to about 64 wt. %, about 50 to about 61 wt. %, about 50 to about 58 wt. %, about 50 to about 55 wt. %; from about 55 to about 90 wt. %, about 55 to about 80 wt. %, about 55 to about 75 wt. %, about 55 to about 70 wt. %, about 55 to about 67 wt. %, about 55 to about 64 wt. %, about 55 to about 61 wt. %; from about 60 to about 90 wt. %, about 60 to about 80 wt. %, about 60 to about 75 wt. %, about 60 to about 70 wt. %; from about 10 to about 90 wt. %, about 10 to about 80 wt. %, about 10 to about 75 wt. %, about 10 to about 70 wt. %, about 10 to about 67 wt. %, about 10 to about 64 wt. %, about 10 to about 61 wt. %, about 10 to about 58 wt. %, about 10 to about 55 wt. %, about 10 to about 53 wt. %, about 10 to about 51 wt. %, about 10 to about 48 wt. %, about 10 to about 45 wt. %, about 10 to about 40 wt. %; from about 70 to about 90 wt. %, about 70 to about 80 wt. %, about 80 to about 90 wt. %, or any range or subrange thereof, based on the total weight of the oral care composition.

In another aspect, the present disclosure provides a method of using the oral care compositions disclosed herein. The methods typically comprise applying any of the compositions as described herein to the teeth, e.g., by brushing, rubbing, swishing when the oral care composition is in the form of a dentifrice, or otherwise administering the oral care compositions to the oral cavity of a subject. The oral care compositions can be administered regularly, such as, for example, one or more times per day.

### EXAMPLES

### Example 1

Six compositions (Examples A-F) were prepared in accordance with the formulations shown in Table 1 (below). Examples A-F were in the form of dentifrices.

TABLE 1

| US INCI Compound | Ex. A | Ex. B | Ex. C | Ex. D | Ex. E | Ex. F |
|---|---|---|---|---|---|---|
| Name | (wt. %) | (wt. %) | (wt. %) | (wt. %) | (wt. %) | (wt. %) |
| Hydrogen Peroxide | 4 | 4 | 4 | 4 | 4 | 4 |
| Sodium | 0.6-2 | 0.6-2 | 0.6-2 | 0.6-2 | 0.6-2 | 0.6-2 |
| Monofluorophosphate | | | | | | |
| Sodium Lauryl Sulfate | 1-4 | 1-4 | 1-4 | 1-4 | 1-4 | 1-4 |
| Powder | | | | | | |
| Tetrasodium | 1-5 | 1-5 | 1-5 | 1-5 | 1-5 | 1-5 |
| Pyrophosphate, and | | | | | | |
| Sodium Acid | | | | | | |
| Pyrophosphate | | | | | | |
| Calcium Pyrophosphate | 10-17 | 10-17 | 10-17 | 10-17 | 10-17 | 10-17 |
| Fumed Silica and | 7-12 | 7-12 | 7-12 | 7-12 | 7-12 | 7-12 |
| Polyvinyl | | | | | | |
| Pyrrolidone | | | | | | |
| KNO₃ | 0 | 0.1 | 1 | 2 | 3 | 5 |
| Butylated | ≤0.1 | ≤0.1 | ≤0.1 | ≤0.1 | ≤0.1 | ≤0.1 |
| Hydroxytoluene | | | | | | |
| Flavoring Agents and | 2-4 | 2-4 | 2-4 | 2-4 | 2-4 | 2-4 |
| Sweeteners (Sodium | | | | | | |
| Saccharin and | | | | | | |
| Sucralose) | | | | | | |
| Water | Q.S. to | Q.S. to | Q.S. to | Q.S. to | Q.S. to | Q.S. to |
| | 100 | 100 | 100 | 100 | 100 | 100 |

### Example 2

Examples A-F were evaluated for stability. Specifically, a sample from each of Examples A-F was filled in a plastic barrier laminate tube and then aged in a temperature controlled environment at a temperature of 60° ° C. for 1 week. The tubes were visually evaluated after the 1-week aging period. The level of active oxygen was also measured before and after the 1-week aging period.

The tubes containing Examples A and B had busted during the 1-week aging period and toothpaste was found outside of the tubes, indicating severe bloating during the aging period. The tubes containing Examples C-E also busted during the 1-week aging period, but the toothpaste remained in the tubes, which suggests that the tubes containing Examples C-E bloated less severely than the tubes containing Examples A and B. The tube containing Example F remained intact during the 1-week aging period, indicating that there was no bloating during the aging period.

The active oxygen level of Example F was measured before and after the aging period to determine the stability of the hydrogen peroxide. Example F had an initial active oxygen level of 4.03% before the 1-week aging period and an active oxygen level of 3.59% after the 1-week aging period. A comparison of Examples A-F revealed that the stability of hydrogen peroxide was significantly enhanced at a higher level of KNO₃, such as at 5 wt. % KNO₃ (see Example F).

1. An oral care composition comprising:
from about 2 to about 10 wt. % of a peroxide source;
about 5 wt. % or more of potassium nitrate; and
about 45 wt. % or more of water,
wherein all weight percentages are based on the total weight of the oral care composition.

2. The oral care composition according to claim 1, wherein the peroxide is selected from hydrogen peroxide, lithium peroxide, potassium peroxide, sodium peroxide, magnesium peroxide, calcium peroxide, barium peroxide, alkyl hydrogen peroxides, diallyl peroxides, alkyl peroxy acids, peroxy esters, diacyl peroxides, benzoyl peroxide, and monoperoxyphthalate, a complex of PVP and hydrogen peroxide, a complex of silica and hydrogen peroxide and a combination of two or more thereof.

3. The oral care composition according to claim 1, wherein the peroxide is hydrogen peroxide.

4. The oral care composition according to claim 1, wherein the potassium nitrate is present in an amount from about 5 to about 15 wt. %.

5. The oral care composition according to claim 1, wherein the water is present in an amount from about 50 to about 75 wt. %.

6. The oral care composition according to claim 1 further comprising:
an anionic surfactant, optionally, in an amount from about 0.5 to about 10 wt. %.

7. The oral care composition according to claim 6, wherein the anionic surfactant is selected from sodium lauryl sulfate, sodium lauryl sarcosinate, sodium cocoyl methyl taurate, sodium monoglyceride sulfate, sodium cetearyl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

8. The oral care composition according to claim 1 further comprising:
a fluoride source in an amount from about 0.1 to about 7 wt. %.

9. The oral care composition according to claim 8, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

10. The oral care composition according to claim 1 further comprising:
a polyol in an amount from about 1 to about 50 wt. %.

11. The oral care composition according to claim 10, wherein the polyol is a humectant selected from sorbitol, glycerin, inositol, maltitol, mannitol, xylitol, propylene glycol, polypropylene glycol (PPG), polyethylene glycol (PEG), a block copolymer of polyoxypropylene and polyoxyethylene, a saccharide, and a combination of two or more thereof.

12-13. (canceled)

14. The oral care composition according to claim 1 further comprising:
a thickening agent in an amount from about 0.1 to about 10 wt. %.

15. The oral care composition according to claim 14, wherein the thickening agent is selected from a silica thickener, a polymer, polysaccharide, and a combination of two or more thereof.

16. The oral care composition according to claim 14, wherein the thickening agent comprises a polymer selected from a polyvinyl pyrrolidone, a polyacrylate, a polymethacrylate, a polyitaconate, an acrylamide, and a combination of two or more thereof.

17. The oral care composition according to claim 14, wherein the thickening agent is selected from carbopol, carboxymethylcellulose, a silica thickener, an amphiphilic polymer polyacrylate crosspolymer-6, and a combination of two or more thereof.

18-19. (canceled)

20. The oral care composition according to claim 1 further comprising: an abrasive comprising calcium pyrophosphate.

21. The oral care composition according to claim 1 further comprising:
a fatty alcohol in an amount from about 0.05 to about 7 wt. %.

22. The oral care composition according to claim 21, wherein the fatty alcohol is selected from decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, isotridecyl alcohol, myricyl alcohol, and a combination of two or more thereof.

23. (canceled)

24. An oral care composition comprising:
from about 2 wt. % to about 10 wt. % of a peroxide source;
about 5 wt. % or more of potassium nitrate;
from about 1 to about 35 wt. % of an abrasive comprising calcium pyrophosphate;
from about 0.5 to about 10 wt. % of an anionic surfactant; and
from about 50 to about 75 wt. % or more of water,
wherein all weight percentages are based on the total weight of the oral care composition.

25. An oral care composition comprising:
from about 2 wt. % to about 10 wt. % of a peroxide source;
from about 1 to about 35 wt. % of an abrasive comprising calcium pyrophosphate;
optionally, from about 0.5 to about 10 wt. % of an anionic surfactant;
a thickening agent selected from a carbopol, carboxymethylcellulose, a silica thickener, polyacrylate crosspolymer-6, and a combination of two or more thereof; and
from about 45 to about 80 wt. % or more of water,
wherein all weight percentages are based on the total weight of the oral care composition.
